(19) United States
(12) Patent Application Publication
(10) Pub. No.: US 2010/0009333 A1
(43) Pub. Date: Jan. 14, 2010

(54) Methods for Acoustic Particle Focusing in Biological Sample Analyzers

(75) Inventor: Robert E. Auer, Key Largo, FL (US)

Correspondence Address: BECKMAN COULTER, INC.;Mitchell E. Alter, P.O. BOX 169015, MAIL CODE 32-A02, MIAMI, FL, 33116-9015, US

(73) Assignee: BECKMAN COULTER, INC., Fullerton, CA (US)

(21) Appl. No.: 12/486,207

(22) Filed: Jun. 17, 2009

Related U.S. Application Data
(60) Provisional application No. 61/079,028, filed on Jul. 8, 2008.

(51) Int. Cl. A01N 1/02; C12Q 1/02; C12N 13/00; B01D 17/00

(52) U.S. Cl. 435/2; 435/29; 435/173.1; 210/748

(57) ABSTRACT

Methods for using acoustic focusing technology on its own or in conjunction with hydrodynamic focusing for analyzing biological samples are provided. In one application, a preferential orientation of biological particles is achieved by applying a substantially elliptical acoustic field. In another application, a sample comprising a fluid medium carrying a plurality of discrete biological particles is pre-concentrated in-line with a sample analyzer, such as a flow cytometer, where a sheath fluid is introduced after acoustic pre-concentration. In a further application, methods for acoustically separating suspended discrete biological particles of different densities from a fluid medium are discussed. The particle-free fluid medium, such as a blood-cell-free and lipid-free clear serum, may be used for chemical analysis.

### CROSS-REFERENCE TO RELATED APPLICATION

This application claims priority to provisional application No. 61/079,028, filed on Jul. 8, 2008, which is hereby incorporated by reference in its entirety.

### BACKGROUND OF THE INVENTION

1. Field of the Invention

The present invention is generally directed to the field of biological sample analyzers. More particularly, it is directed to application of acoustic focusing technology in biological sample analyzers.

2. Background Art

Biological sample analyzers, such as, flow cytometers and hematology instruments, are widely used for clinical and research use. A biological sample may comprise a fluid medium carrying a plurality of discrete biological particles, e.g. cells or microsphere substrates, suspended therein. Information obtained from the biological particles is often used for clinical diagnostics and/or other analyses. For example, in a flow cytometer, discrete biological particles, e.g. cells or microsphere substrates, suspended in a fluid medium, are focused preferably in a single-file configuration in a flow path by some focusing means. The focused biological particles sequentially pass through a spatially localized excitation point along the flow path in a flow cell. The passage of the focused biological particles through the spatially localized excitation point generates signals that are collected by a sensor having one or more detectors positioned proximal to the flow path. For example, in a flow cytometer, the spatially localized excitation may be a laser radiation shone at a particular point along the flow path, and the collected signal may be light scattered by the biological particles, and laser-induced radiations emitted from fluorescently-tagged internal and/or surface components of the biological particles.

Many conventional biological sample analyzers use hydrodynamic focusing as the focusing means, where a sheath fluid is introduced to entrain the fluid medium and to constrain the biological particles along approximately a centerline of the flow path. In other words, the sheath fluid creates a “virtual aperture” along a cross-sectional plane of the flow path, wherein an annulus comprising the sheath fluid surrounds a core comprising the biological sample, with the focused biological particles suspended in the fluid medium.

The velocity of the biological sample in the flow path can be set by a pressure applied to the sheath fluid. If the pressure on the sheath fluid is set too high in an attempt to flow the biological sample at a higher velocity to obtain higher throughput, then the flow through the sensors can become non-laminar. A non-laminar flow results in the biological particles traversing the flow path through the sensor via random paths yielding inaccurate measurements. On the other hand, if a pressure on the fluid medium of the biological sample is increased with respect to the pressure on the sheath fluid, such that the biological sample flow becomes a larger percentage of the total cross section of the flow through the sensor, then it becomes difficult to maintain a constant diameter of the biological sample flow in the center of the sheath fluid, and the biological particles may steer away from the centerline of the flow path. An inconsistent sample flow reduces the resolution of the measurement and degrades the ability of the system to differentiate between various biological particle types. In addition to the requirement of sheath pressure control, in hydrodynamic focusing, it may be necessary to match viscous properties of the sheath fluid and the fluid medium of the biological sample to achieve better velocity regulation. Thus, as discussed above, the throughput of a biological sample analyzer using hydrodynamic focusing is limited by various factors, including, but not limited to, applied pressure, properties of the fluid medium and the sheath fluid, cross-sectional dimension of the flow path, etc.

Acoustic focusing has been used as an alternative focusing means to circumvent at least some of the limitations of hydrodynamic focusing, thereby offering potentially higher throughput. In a system using acoustic focusing, a radio frequency electrical signal is applied to a transducer coupled to a tube through which the biological sample flows. The electrical signal creates an acoustic field distribution within the tube, aligning the biological particles along certain points or localized regions along the cross section of the tube, thereby achieving focusing of the biological particles. U.S. Pat. No. 7,340,957 to Kaduchak et al. discusses an example application of acoustic focusing in flow cytometry, where no sheath fluid is used. Acoustic focusing technology depends on the resonant frequency of the tube structure, which in turn is dependent on the material and dimensions of the tube, the length of the acoustic transducer, etc. Additionally, a constant monitoring of applied acoustic field may be necessary to maintain a resonant frequency and to compensate for ambient temperature fluctuations.

It is desirable to regulate the velocity of the biological sample along the flow path for efficiently focusing the biological particles, maintaining the focusing throughout the flow path through the sensor, and collecting the generated signals by the detectors included in the sensor, thereby achieving an optimum throughput. What is needed are versatile systems and methods that can combine the desirable features of both acoustic and hydrodynamic focusing, as required, while avoiding the shortcomings of each of the focusing approaches. The systems should be configured to selectively adopt just one of the acoustic and hydrodynamic focusing means, or both means in conjunction, depending on the end application.

### BRIEF SUMMARY OF THE INVENTION

Embodiments of the present invention provide various methods that use acoustic focusing technology for biological sample analysis applications. Such methods may be used, for example, in cytometry and/or hematology instruments.

One embodiment of the present invention provides a method for orienting biological particles in a biological sample analyzer, where each of the biological particles has at least one face with a substantially planar region. The method comprises: establishing a flow of a fluid medium within a sample inlet tube, the fluid medium carrying the discrete biological particles; and, applying a substantially elliptical acoustic field having a major axis along a direction of a maximum acoustic field strength in a cross-sectional plane of the sample inlet tube, to focus and preferentially orient the biological particles, such that a respective face of each of the biological particles is oriented normal to the major axis. The method may be used to measure signals from the biological particles. The method may be used for measuring and sorting sperm cells. The method may also be used to measure signals from red blood cells.

Another embodiment of the present invention provides a method for in-line pre-concentration of a sample comprising a fluid medium carrying a plurality of discrete biological particles. The method comprises: applying an acoustic field along a length of a sample inlet tube that receives the sample, thereby acoustically focusing the biological particles in the sample inlet tube, wherein the sample inlet tube is positioned in-line with a subsequent hydrodynamically focused flow cell; removing a substantial portion of the fluid medium from the periphery of the sample inlet tube, leaving the acoustically focused biological particles carried by a remaining portion of the fluid medium in a central region of the sample inlet tube, thereby pre-concentrating the sample; directing the pre-concentrated sample comprising the fluid medium and the acoustically focused biological particles to the flow cell; and, introducing a sheath fluid in the flow cell, the sheath fluid creating an annular aperture within the flow cell surrounding the fluid medium carrying the acoustically focused biological particles in a central region of the flow cell.

A further embodiment of the present invention provides a method for separating suspended discrete, higher density biological particles from a lower density fluid medium that carries the biological particles. The method comprises: focusing the biological particles along a sample inlet tube by applying an acoustic field along a length of the sample inlet tube that receives the fluid medium with the suspended biological particles through an input end; and, dividing an output end of the sample inlet tube into a first and second output channels, wherein the first output channel collects a relatively smaller portion of the fluid medium including the acoustically focused biological particles from a central region of the sample inlet tube, and the second output channel collects a relatively larger remaining portion of the fluid medium substantially free of the acoustically focused biological particles from a peripheral region of the sample inlet tube. The method further comprises: directing the portion of the fluid medium substantially free of the acoustically focused biological particles towards an inlet of a chemical analyzer. The method may be used in clinical diagnostics, e.g. serum chemistries.

Yet another embodiment of the present invention provides a method for separating lower density lipid particles from a lipemic higher density serum that is free of blood cells. The method comprises: applying an acoustic field along a length of a sample inlet tube to acoustically drive the lipid particles towards a peripheral region of the sample inlet tube, leaving a lipid-free clear serum in a central region of the sample inlet tube; and dividing an output end of the sample inlet tube into a first and second output channels, wherein the first output channel collects a portion of the serum including the acoustically-driven lipid particles from the peripheral region of the sample inlet tube, and the second output channel collects the lipid-free clear serum from the central region of the sample inlet tube. The lipid-free clear serum may be used for chemical analysis and/or clinical diagnostics.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the present invention and, together with the description, further serve to explain the principles of the invention and to enable a person skilled in the relevant art(s) to make and use the invention.

FIG. 1 illustrates a portion of a flow chamber included in a conventional system for analyzing and sorting biological samples that uses hydrodynamic focusing.

FIG. 2 illustrates a modified flow chamber where both acoustic and hydrodynamic focusing are used, according to an embodiment of the present invention.

FIG. 3 illustrates a first mode of operation of a biological sample analyzer, according to an embodiment of the present invention.

FIG. 4 illustrates a second mode of operation of a biological sample analyzer, according to an embodiment of the present invention.

FIG. 5 schematically shows a beveled output end of a sample inlet tube for preferentially orienting a sperm cell, according to a conventional method.

FIGS. 6A and 6B illustrate front view and side view, respectively, of red blood cells, that may be preferentially oriented in a biological sample analyzer using an elliptical acoustic field, according to an embodiment of the present invention.

FIG. 7 illustrates a cross section of a sample inlet tube, schematically showing an elliptical acoustic field distribution, according to an embodiment of the present invention.

FIGS. 8-11 illustrate flowcharts depicting exemplary methods of applications of acoustic focusing technology in biological sample analyzers, according to various embodiments of the present invention.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION OF THE INVENTION

Embodiments of the present invention provide applications of acoustic focusing technology on its own or in conjunction with hydrodynamic focusing for analyzing biological samples. In the detailed description that follows, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

It is also noted that in the subsequent description, the term “biological sample analyzer” encompasses a variety of instruments, including, but not limited to, particle analyzers, such as flow cytometers, and chemical analyzers, such as, serum measurement instruments.

### Example System Configuration and Modes of Operation

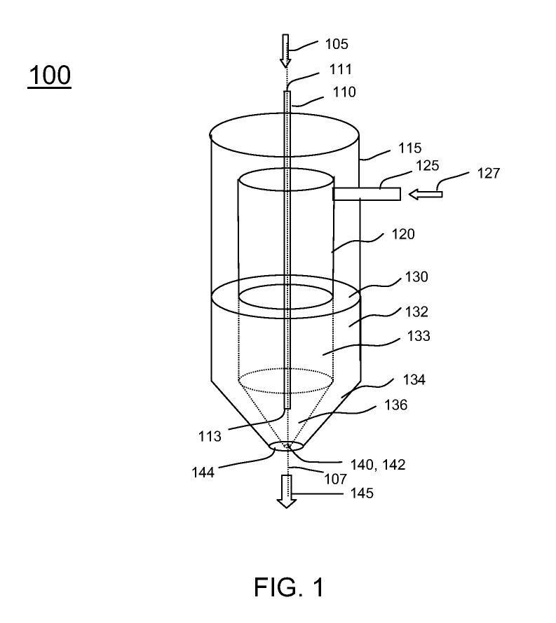

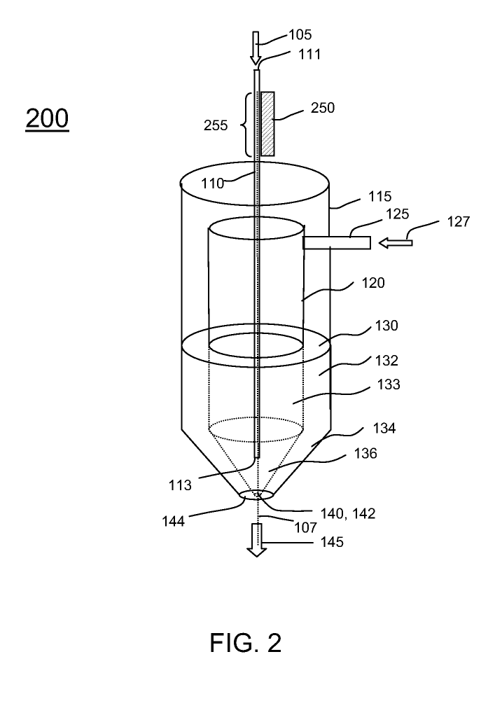

Embodiments of the present invention may combine both acoustic focusing and hydrodynamic focusing in series or in parallel. FIG. 1 illustrates a portion of a flow chamber 100 included in a conventional system for analyzing and sorting biological samples that uses hydrodynamic focusing. FIG. 2 illustrates a flow chamber 200, which is a modified version of flow chamber 100, and is configured to use both acoustic focusing and hydrodynamic focusing. However, modified flow chamber 200 may selectively use only acoustic focusing, or only hydrodynamic focusing, or both, depending on the application.

In the example embodiment discussed with reference to FIG. 2, acoustic focusing can be used to focus and/or pre-concentrate a biological sample before the biological sample is directed to a flow cell of a flow cytometer. As discussed in the “Background Art” section, acoustic focusing has the potential to increase overall, throughput of a biological sample analyzer, such as a flow cytometer. Though using a sheath fluid may present some limitations in terms of the throughput achievable by a biological sample analyzer, there are certain advantages of using a sheath fluid, which a sheathless acoustic focusing device cannot offer. For example, the sheath fluid creates a protective environment around a core of the biological sample, preventing the biological particles and the fluid medium of the biological sample from direct contact with the walls of the sample inlet tube, thus, minimizing contamination probability. By using a sheath fluid and regulating the pressure applied on the sheath fluid, it is possible to better maintain the alignment of the acoustically-focused biological particles, thus relaxing some of the acoustic field monitoring requirement to adapt to environmental fluctuations.

Flow chamber 100 of FIG. 1 comprises a sample inlet tube 110 having an input end 111 and an output end 113, a flow chamber housing 115, a sheath fluid chamber 120 including a sheath fluid inlet 125, and a nozzle 130 with an output orifice 140.

A biological sample 105 is introduced through input end 111 of sample inlet tube 110, which is disposed partially within flow chamber housing 115. Biological sample 105 comprises a fluid medium and biological particles suspended therein. For example, biological sample 105 may comprise whole blood with red blood cells, white blood cells, and other cells and/or particles, such as lipid particles, suspended in blood serum. Biological sample 105 may also be a pre-processed sample, such as, a selectively lysed blood sample, primarily having white blood cells. Biological sample 105 may be diluted, or pre-concentrated using a centrifuge or other means. Example biological sample 105 may include a fluid medium carrying sperm cells or other type of cells or particles. Persons skilled in the art will appreciate that embodiments of the present invention are not limited to the examples discussed above, and even non-biological samples may be analyzed by the embodiments of the present invention. The fluid medium is sometimes referred to as “supernatant.”

A sheath fluid 127 is introduced through sheath fluid inlet 125 to sheath fluid chamber 120. As shown in FIG. 1, sheath fluid chamber 120 is disposed within flow chamber housing 115. Nozzle 130 is coupled to flow chamber housing 115. Flow chamber housing 115, sample inlet tube 110, and sheath fluid chamber 120 have a common central axis 107 passing through output orifice 140 of nozzle 130. Nozzle 130 has a uniform-diameter upper portion 132 coupled to a gradually tapering lower portion 134 ending at output orifice 140. Sheath fluid chamber 120 also has a uniform-diameter upper portion 133 coupled to a gradually tapering lower portion 136 ending in an output orifice 142. Sheath fluid chamber 120 is at least partially disposed within nozzle 130 in such a way that output orifice 140 of nozzle 130 and output orifice 142 of sheath fluid chamber 120 coincide on a same output plane 144.

Output end 113 of sample inlet tube 110 is disposed within gradually tapering lower portion 136 of sheath fluid chamber 120. Hydrodynamic focusing of suspended biological particles approximately along central axis 107 is realized within gradually tapering lower portion 136 of sheath fluid chamber 120. Gradually tapering lower portion 136 may be an integrated part of a flow cell or is coupled to a flow cell that includes a localized excitation point and a sensor region (not shown). Dimensions of gradually tapering lower portion 136 may be carefully designed to achieve an effective hydrodynamic focusing. Output flow 145 coming out of output orifices 140 and 142 comprises a core comprising biological sample 105 with the biological particles focused approximately along central axis 107, surrounded by an annulus of sheath fluid 127. Output flow 145 is directed to the sensor region (not shown) of the flow cell where signals generated by the biological particles are collected by the detectors and analyzed further. Other examples of conventional biological sample and chemical analyzer systems are known to those skilled in the art and include systems which analyze the biological particle or microsphere substrates by one or more parameters of light, florescence, direct current (volume) and radio frequency. Some of these systems are currently sold by Beckman Coulter, Inc.; Becton Dickinson and Company; Sysmex, Inc.; and Abbott Laboratories, Inc.

An example conventional biological sample analyzer system that may use a component such as flow chamber 100 described in FIG. 1 is referred to as TTM (Triple Transducer Module), manufactured by Beckman Coulter. TTM systems are widely used for mid-range and high-end hematology analyzers. A TTM system may use a technology such as VCS (Volume, Conductivity, Scatter) measurement technology, also by Beckman Coulter. TTM systems in conjunction with selective lysis and in some cases absorptive stains may yield five-part differentials, reticulocyte counts, and nucleated red blood cell counts. All of the above measurements require mixing of an aliquot of blood with a series of reagents, which dilute that blood aliquot by approximately a factor to ten or more. Since a diluted sample is introduced into the TTM system, it may not be possible to obtain a high throughput without some means of pre-concentration of the diluted sample. It may be possible to replace a sample injection mechanism of a TTM system with a sample inlet tube fitted with an acoustic transducer in order to improve overall throughput.

FIG. 2 illustrates modified flow chamber 200, where an acoustic transducer 250 is coupled to sample inlet tube 110. An example of such a transducer is available, for example, from Acoustic Cytometry Systems, of Los Alamos, N. Mex. Sample inlet tube 110 may have any shape, including but not limited to a uniform-diameter-circular-cross-section cylindrical shape. Acoustic transducer 250 may be a linear transducer coupled tangentially to a single line along a length 255 of sample inlet tube 110. Other types of configurations of acoustic transducer 250 may be used without departing from the scope of the present invention. For example, a transducer that substantially conforms to the cross-sectional shape of sample inlet tube 110 may be used. If sample inlet tube 110 is cylindrical, acoustic transducer 250 may have a hollow cylindrical portion that wraps around a length 255 of sample inlet tube 110. In another example, transducer 250 may have one or more pairs of planar plates disposed around sample inlet tube 110. Persons skilled in the art will appreciate that a piezoelectrically driven transducer (“PZT drive”) or other types of transducers may be used.

Acoustic transducer 250 creates an acoustic field distributed along the cross section of sample inlet tube 110. Excitation frequencies generated by acoustic transducer 250 may be in the kHz or MHz range, depending on the configurations of different systems. As would be known to persons skilled in the art, excitation frequency for a system is chosen based on various factors, including, but not limited to, material and diameter of the sample inlet tube, length of the transducer, density of the fluid medium, density of the biological particles, etc. Biological particles suspended in the fluid medium are acoustically driven to certain locations within the cross section of sample inlet tube 110 depending on the positions of the acoustic nodes and respective density of the biological particles. Higher-density particles, such as, blood cells, tend to align approximately along a centerline of sample inlet tube 110, while relatively lower-density particles, such as, lipid particles, tend to be driven toward the walls of sample inlet tube 110. It is possible to pre-concentrate biological sample 105 by getting rid of excess fluid medium from peripheral regions of sample inlet tube 110 before directing the biological sample with acoustically-focused particles towards a flow cell of a biological sample analyzer.

In a particle analyzer system that uses modified flow chamber 200 shown in FIG. 2, hydrodynamic focusing may be utilized to maintain the alignment of the acoustically-focused biological particles. The use of hydrodynamic focusing subsequent to acoustic focusing provides additional flexibility in regulating the velocity of sample flow through the sensor region of the flow path. It is possible to attain an increased throughput from a biological sample analyzer that uses modified flow chamber 200 compared to the throughput achieved by a conventional biological sample analyzer that uses conventional flow chamber 100.

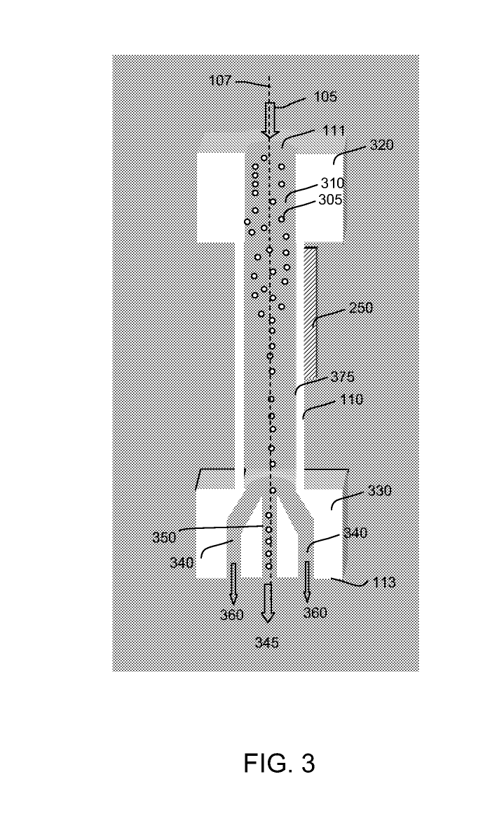

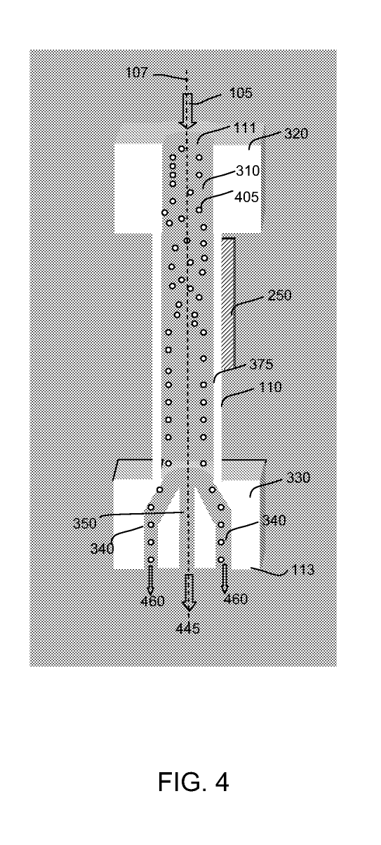

Persons skilled in the art will appreciate that input end 111 and output end 113 of sample inlet tube 110 may be modified depending on modes of operation and/or end applications. Input end 111 and output end 113 have respective extension regions 320 and 330, as shown in FIGS. 3 and 4. Extension regions 320 and 330 may assist in coupling sample inlet tube 110 to other components along the flow path. Additionally, input end 111 of sample inlet tube 110 may comprise more than one input channels (not shown in FIGS. 3 and 4). Similarly, output end 113 of sample inlet tube 110 may be divided into two or more output channels 340 and 345, as shown in FIGS. 3 and 4. Embodiments of the present invention may be operated in at least two modes. A first mode, known as “Concentrate Mode,” may be used for concentrating higher-density biological particles in a central region of sample inlet tube 110, as shown in FIG. 3. A second mode, known as “Purge Mode,” may be used for removing lower-density biological particles from fluid medium in order to obtain a particle-free fluid medium or clear supernatant, as shown in FIG. 4. It is to be appreciated that “Concentrate Mode” and “Purge Mode” of operations may be carried out in series to obtain a clear supernatant that is free of both high-density and low-density biological particles. A single transducer 250 may be used at the same or different frequencies for the “Concentrate Mode” and the “Purge Mode” of operations. Two or more transducers may be employed to run the “Concentrate Mode” and “Purge Mode” of operations in parallel, without departing from the scope of the present invention.

In FIG. 3, the “Concentrate Mode” of operation is illustrated by showing a flow path along a longitudinal slice of sample inlet tube 110. This mode is useful when initial biological sample 105 being introduced to sample inlet tube 110 comprises a plurality of high-density biological particles 305 suspended in fluid medium 310. Lower-density biological particles (not shown in FIG. 3) may or may not be present in biological sample 105. Acoustic transducer 250 is coupled to a portion of a wall 375 of sample inlet tube 110. Acoustic transducer 250 applies an acoustic field to sample inlet tube 110, aligning high density particles 305 along central axis 107 of sample inlet tube 110. A portion of fluid medium 310 is routed to peripheral output channel 340, leaving a smaller portion of fluid medium 310 containing the acoustically focused high-density biological particles 305 to be routed to central output channel 350. Flow 360 coming out of peripheral output channel 340 may be disposed off as waste, or is used as the initial sample for a subsequent “Purge Mode.” Flow 360 may also be directed to an inlet of a chemical analyzer (not shown) for measurement of chemical properties of fluid medium 310. Flow 345 coming out of central output channel 350 may be directed to the flow cell of a particle analyzer, such as a flow cytometer.

In FIG. 4, the “Purge Mode” of operation is illustrated by showing a flow path along a longitudinal slice of sample inlet tube 110. This mode is useful when initial biological sample 105 being introduced to sample inlet tube 110 comprises a plurality of low-density biological particles 405 suspended in fluid medium 310. Higher-density biological particles (not shown in FIG. 4) are already separated from initial biological sample 105. Acoustic transducer 250 applies an acoustic field to sample inlet tube 110, driving low-density particles 405 towards wall 375 of sample inlet tube 110, leaving a particle-free clear supernatant or fluid medium 310 in the central region of sample inlet tube 110. A portion of fluid medium 310 containing the low-density biological particles 405 is routed to peripheral output channel 340, while the remaining portion of the particle-free fluid medium 310 is routed to central output channel 350. Flow 460 coming out of peripheral output channel 340 may be disposed as waste. Flow 445 coming out of central output channel 350 may be directed to an inlet of a chemical analyzer (not shown) for measurement of chemical properties of fluid medium 310.

Persons skilled in the art will appreciate that the present invention is not limited to the operational modes discussed above.

In the subsequent sections, various applications of acoustic focusing technology are discussed, according to embodiments of the present invention.

Orientation Control of Biological Particles using Acoustic Focusing

In a flow-through method of biological particle analysis, such as the methods used by a flow cytometer, the resolution and efficacy of signal measurement can be improved by optimally orienting the biological particles as they traverse the localized excitation point. Orientation control has less benefit if the biological particles are primarily irregular-shaped or spherical-shaped, such as some white blood cells. However, if the population of the biological particles is known to primarily comprise biological particles of a predictable shape, each particle having at least one face with a substantially planar region, then a preferential orientation of the biological particles can vastly improve signal collection and measurement. For example, a biological particle may be substantially disk-shaped, wherein an area of at least one face of the biological particle is relatively larger than an area of a side surface thereof. Persons skilled in the art will appreciate that the term “disk-shaped” encompasses a variety of shapes, including, but not limited to, a flattened bulb-like shape, a flattened ovoid shape, a biconcave shape, a biconvex shape, a concave-convex shape, etc.

An example application of the present invention may be for analyzing and/or sorting sperm cells using a flow cytometer configured to have an acoustic transducer. For example, a flow cytometer of the present invention may have a modified flow chamber 200, as described in FIG. 2. This application can be used, for example, in the animal husbandry and breeding industry. Because of the differences in the DNA content of the X and Y chromosomes, sperm cells containing either X or Y chromosome, can be separated from one another based on accurate measurement of total DNA content. It has been known that accurate measurement of DNA content in a sperm cell is dependent on the orientation of the sperm with respect to the localized excitation point and fluorescence detection system in the flow cytometer. Because sperm cells are disk-shaped (i.e., flattened ovoid shaped) rather than spherical, their optical properties vary with orientation. In a flowing system, they orient along the axis of flow but can have any orientation around the axis of flow. Accuracy of measurement of DNA content can be improved if signals are collected through the planar surfaces of the cells.

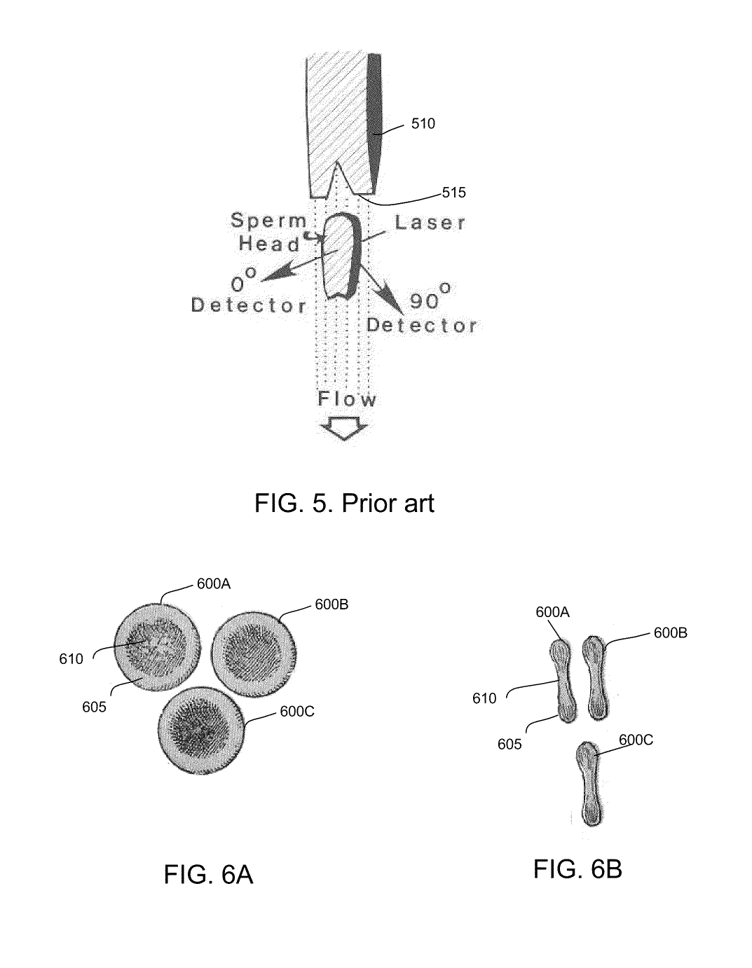

In conventional systems, hydrodynamic focusing has been used to preferentially orient the sperm cells via either a beveled output end of the sample inlet tube and/or via an elliptical nozzle. For example, FIG. 5 schematically shows a beveled output end 515 (also referred to as a beveled tip) of a sample inlet tube 510 designed for preferentially orienting a head portion of the sperm cells, according to a method described by L. A. Johnson of US Department of Agricultural Research Service, Beltsville, Md., and D. Pinkel of Lawrence Livermore National Laboratories, Livermore, Calif., in a paper titled, “Modification of a Laser-Based Flow Cytometer for High-Resolution DNA Analysis of Mammalian Spermatozoa,” published in the journal Cytometry, vol. 7, no. 3, in May 1986, pp. 268-273. As shown in FIG. 5, the beveled output end 515 preferentially orients the head portion of the sperm cells so that they are illuminated on one of their approximately flat surfaces by a laser beam, and a 0° detector and a 90° detector collect fluorescence signals from the sperm nucleus. Other types of beveled nozzle shapes for hydrodynamic orientation control are described in U.S. Pat. No. 6,263,745 by Buchanan et al.

Types of disk-shaped biological particles that can be preferentially oriented include red blood cells. FIG. 6A shows three red blood cells 600A-C, viewed from the front, each having a face 605 with a substantially planar region 610. FIG. 6B shows side view of the red blood cells 600A-C. Though in the side view, red blood cells are seen as having biconcave profiles, it still can be considered that their respective faces 605 are substantially planar.

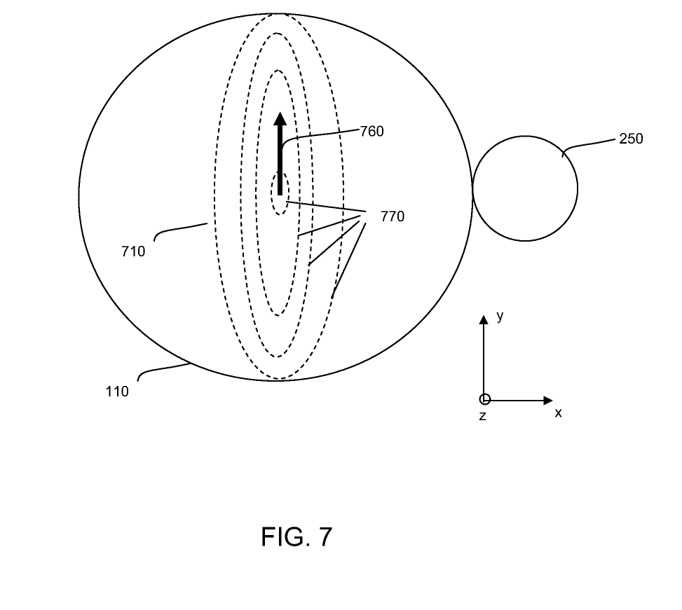

Embodiments of the present invention use a substantially elliptical acoustic field to achieve preferential orientation of substantially disk-shaped biological particles. The modified flow chamber 200 shown in FIG. 2 may be used for preferentially orienting the biological particles. FIG. 7 illustrates a cross section of sample inlet tube 110, schematically showing an elliptical acoustic field distribution 710. Acoustic field 710 is induced by a linear transducer 250, which is coupled tangentially to sample inlet tube 110. Dashed lines 770 indicate substantially elliptical equipotential acoustic field lines. As shown in FIG. 7, acoustic field 710 is substantially stronger in y direction than it is in the x direction. The major axis indicated by the thick arrow 760 indicates the direction of the strongest acoustic field. Elliptical acoustic field 710 orients disk-shaped biological particles in a way such that a respective substantially planar face of each of the biological particles is oriented normal to major axis 760, as the biological particles flow along the z direction towards the flow cell. This preferential orientation of the disk-shaped biological particles ensures efficient interaction with the localized excitation point, as well as maximization of signal collection by the detectors, which are also pre-aligned to the elliptical acoustic field 710.

The acoustic orientation control approach offers several advantages over the current hydrodynamic orienting approaches. It has been found that the hydrodynamically oriented systems using only a beveled tip 515 works effectively at relatively low flow rates. This is because the orientation takes place locally at the beveled tip 515. If the flow of sample 105 is increased, a blooming of the sample flow at the beveled tip 515 may negate the orienting effect. Additionally, hydrodynamic impulse forces may damage the biological particles. In contrast, a system using acoustic focusing applies the orienting force at a lower consistent level over a longer distance of sample introduction tube 110. Thus, a system using acoustic focusing is likely to be less sensitive to sample flow rate and less damaging to the biological particles. As shown in FIG. 2, acoustic focusing may be used in conjunction with hydrodynamic focusing to improve and/or maintain the acoustically-achieved focus and orientation of the biological particles.

### Method for Preferentially Orienting Biological Particles

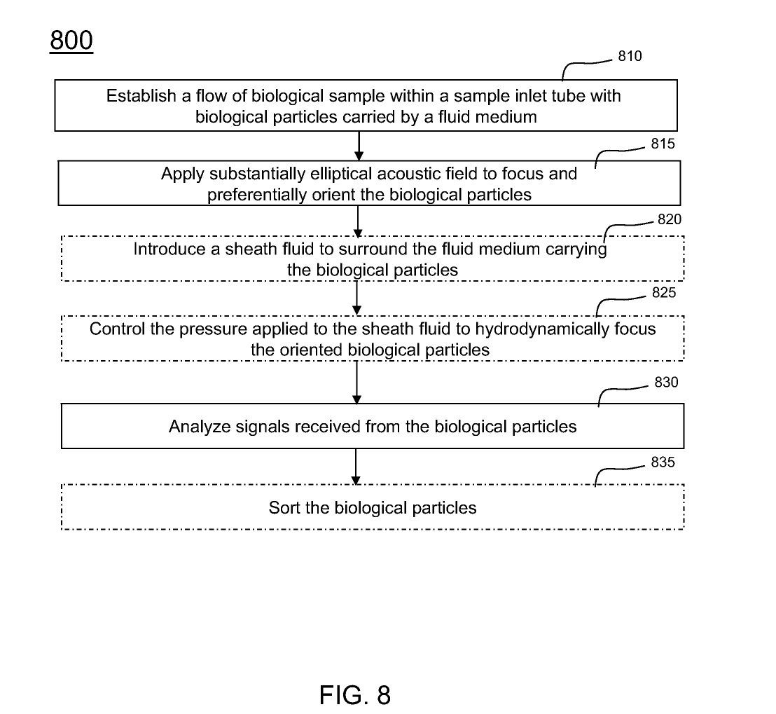

FIG. 8 schematically illustrates a flowchart of a method 800 for analyzing preferentially oriented biological particles in a biological sample analyzer, according to an embodiment of the present invention. In one example, method 800 can be practiced by one or more of the systems discussed above.

Method 800 starts at step 810, wherein a flow of biological sample is established within a sample inlet tube. The fluid flow comprises a fluid medium and discrete biological particles suspended therein. As mentioned above, the biological particles may be substantially disk-shaped. Though method 800 is not limited to disk-shaped biological particles, method 800 offers certain advantages by orienting disk-shaped biological particles.

In step 815, a substantially elliptical acoustic field is applied to focus and preferentially orient the biological particles, approximately along a centerline of the sample inlet tube, such that a respective face of each of the biological particles is oriented normal to a the direction of maximum field strength of the substantially elliptical acoustic field.

Steps 820 and 825 are optional steps (indicated by dashed lines) if both acoustic and hydrodynamic focusing techniques are used, as discussed with respect to FIG. 2. In step 820, a sheath fluid is introduced to surround the fluid medium carrying the biological particles. In step 825, a pressure applied to the sheath fluid is controlled to further hydrodynamically focus the acoustically focused biological particles.

In step 835, signals received from the biological particles are measured and analyzed.

In another optional step 840, the biological particles are sorted based on the analysis done in step 835. For example, sperm cells containing X chromosomes may be sorted separately from sperm cells containing Y chromosomes.

### Method for In-Line Pre-Concentration of a Biological Sample

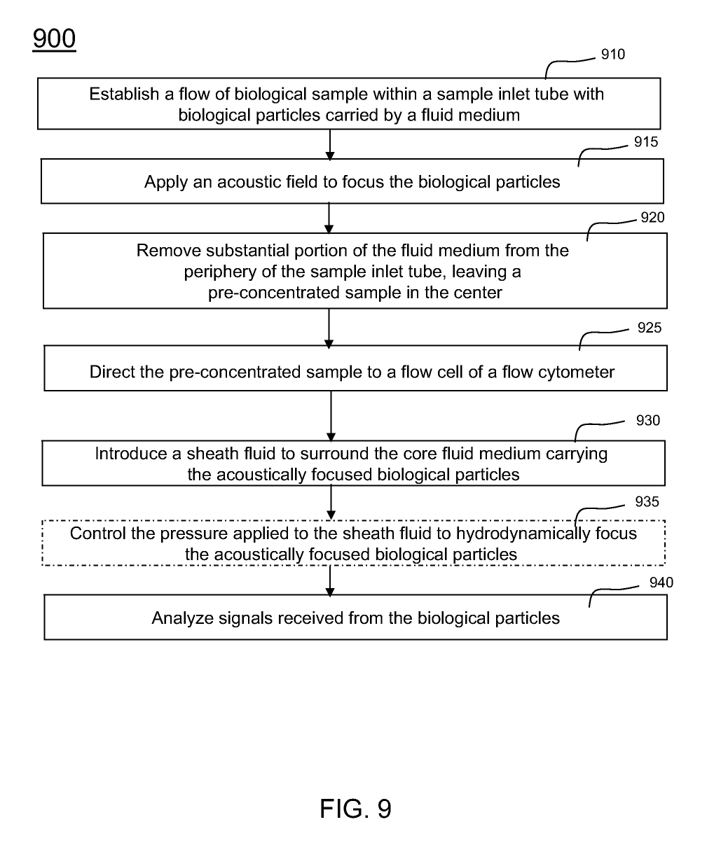

FIG. 9 schematically illustrates a flowchart of a method 900 for in-line pre-concentration of a biological sample comprising a fluid medium carrying a plurality of discrete biological particles, according to an embodiment of the present invention. In one example, method 900 can be practiced by one or more of the systems discussed above, such as modified flow chamber 200 described in FIG. 2, and the system described in FIG. 3 operating in a “Concentrate Mode.”

Method 900 starts at step 910, wherein a flow of biological sample is established within a sample inlet tube positioned in-line with a flow cell of a biological sample analyzer, such as a flow cytometer. The fluid flow comprises a fluid medium and discrete biological particles suspended therein. For example, the biological sample may be whole blood, or it can be diluted lysed blood.

In step 915, an acoustic field is applied to focus the biological particles, approximately along a centerline of the sample inlet tube. As discussed in method 800, if intended, biological particles may be preferentially oriented in step 915.

In step 920, a substantial portion of the fluid medium is removed from the periphery of the sample inlet tube, leaving the acoustically focused biological particles carried by a remaining portion of the fluid medium in a central region of the sample inlet tube, thereby pre-concentrating the sample.

In step 925, the pre-concentrated sample is directed towards to the flow cell of the biological sample analyzer.

In step 930, a sheath fluid is introduced in the flow cell, the sheath fluid creating an annular aperture within the flow cell surrounding the fluid medium carrying the acoustically focused biological particles in a central region of the flow cell.

In optional step 935, a pressure applied to the sheath fluid is controlled to further hydrodynamically focus the acoustically focused biological particles. Note that the sheath fluid can be used only to create a protective environment around the acoustically focused sample without contributing actively to further hydrodynamic focusing of the acoustically-focused sample. For example, in certain embodiments, applied pressure on the sheath fluid does not have to be controlled actively, if hydrodynamic focusing is not actively pursued. However, even if no active hydrodynamic focusing is being used, it is desirable to use a sheath fluid whose refractive index matches with the refractive index of the fluid medium, because light signals coming out of the biological particles pass through both the fluid medium and the sheath fluid to reach the detectors, and a refractive index mismatch may direct the light signals away from the detectors.

In step 940, signals received from the biological particles are measured and analyzed.

Method for Separation of Suspended Biological Particles from a Fluid Medium

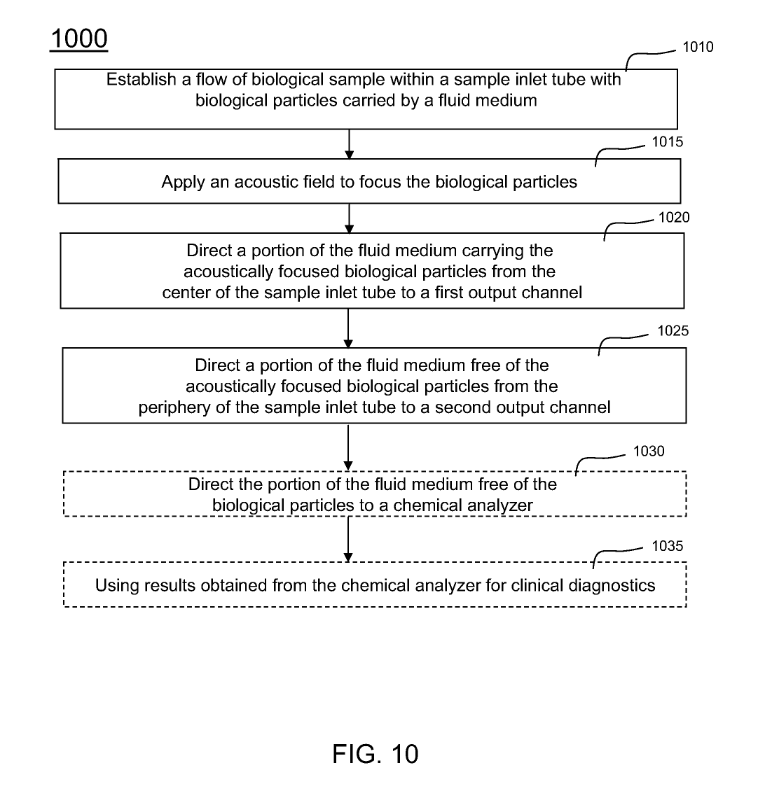

FIG. 10 schematically illustrates a flowchart of a method 1000 for separating suspended discrete biological particles from a fluid medium that carries the biological particles, according to an embodiment of the present invention. In one example, method 1000 can be practiced by one or more of the systems discussed above, such as the system described in FIGS. 3 and 4.

Method 1000 starts at step 1010, and goes to step 1015 thereafter. Steps 1010 and 1015 are identical to steps 910 and 915, respectively, as discussed in method 900.

In step 1020, a portion of the fluid medium carrying the acoustically focused biological particles is directed from the center of the sample inlet tube to a first output channel.

In step 1025, the remaining portion of the fluid medium free of the acoustically focused biological particles is directed from the periphery of the sample inlet tube to a second output channel. Thus, at least high-density biological particles are separated from the fluid medium flowing in the second output channel. Additional low-density particles may still be suspended in the fluid medium, and it may be required to run the fluid medium through the sample inlet tube once more for a follow-up “Purge Mode” operation. Alternatively, a second acoustic field may be applied to the second output channel further downstream to acoustically drive the additional low-density particles towards a peripheral region of the second output channel. The output end of the second output channel may be divided into a third and fourth output channels, wherein the third output channel collects a portion of the fluid medium including the acoustically-driven additional low-density particles from the peripheral region of the second output channel, and the fourth output channel collects a remaining portion of the fluid medium from the central region of the second output channel.

In optional step 1030, the fluid medium free of at least the acoustically-focused high-density particles is directed towards an inlet of a chemical analyzer. If both high-density and low-density particles are removed from the fluid medium, then only the particle-free clear fluid medium or supernatant is directed towards the chemical analyzer.

In optional step 1035, results obtained from the chemical analyzer may be used for clinical diagnostics or other analytical purposes.

Method for Separation of Lipid Particles from a Lipemic Serum

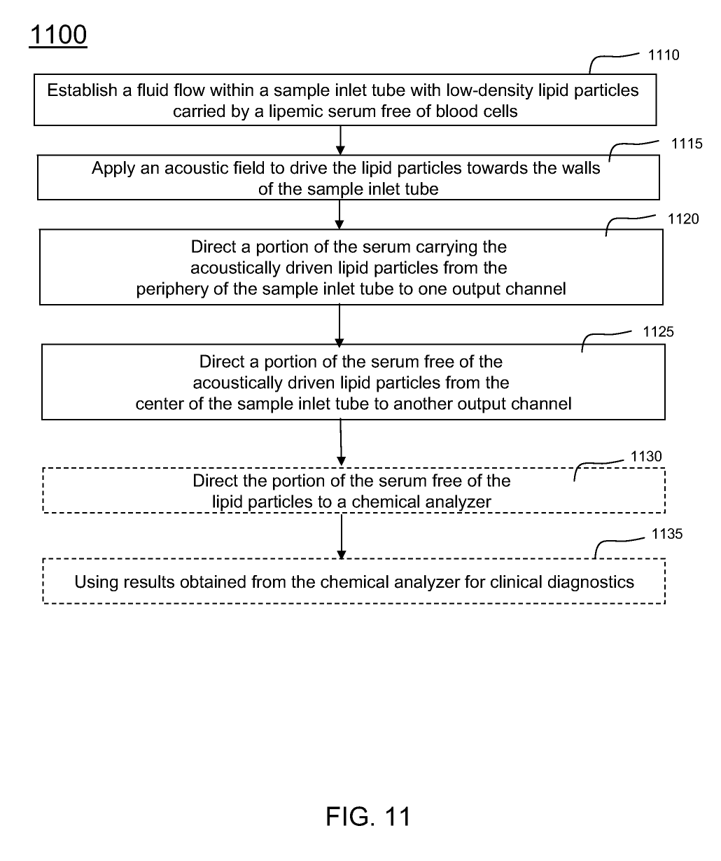

FIG. 11 schematically illustrates a flowchart of a method 1100 for separating suspended low-density lipemic particles from a lipemic serum sample, according to an embodiment of the present invention. In one example, method 1100 can be practiced by one or more of the systems discussed above, such as the system described in FIG. 4, operating in “Purge Mode.”

In step 1110, a fluid flow is established in a sample inlet tube, wherein the fluid flow comprises lipemic serum with suspended lipid particles in it. Higher density particles, such as blood cells have been previously removed from the sample.

In step 1115, an acoustic field is applied to drive the lipid particles towards the walls of the sample inlet tube. Generally, a different or same acoustic frequency is used to drive the lipid particles out of the lipemic serum than what is used to separate higher-density blood cells.

In step 1120, a portion of the serum carrying the acoustically-driven lipid particles is directed from the periphery of the sample inlet tube to a peripheral output channel.

In step 1125, the remaining portion of the serum free of the acoustically-driven lipid particles is directed from the center of the sample inlet tube to a central output channel.

In optional step 1130, the lipid-free serum from the central output channel is directed towards a chemical analyzer.

In optional step 1135, results obtained from the chemical analyzer may be used for clinical diagnostics or other analytical purposes.

The steps of the flowcharts showing methods 800-1100 are for illustrative purpose only, and do not have to take place in the order shown. There may be additional intermediate or end steps that are not shown in the flowcharts showing methods 800-1100. Some of the steps may be optional, and/or specific to particular embodiments of the present invention.

Persons skilled in the art will appreciate that the four application methods discussed above are for illustrative purposes, and the present invention is not limited to those four applications only.

The foregoing description of the invention has been presented for purposes of illustration and description. It is not intended to be exhaustive or to limit the invention to the precise form disclosed, and other modifications and variations may be possible in light of the above teachings. The embodiments were chosen and described in order to best explain the principles of the invention and its practical application to thereby enable others skilled in the art to best utilize the invention in various embodiments and various modifications as are suited to the particular uses contemplated. It is intended that the appended claims be construed to include other alternative embodiments of the invention except insofar as limited by the prior art.

What is claimed is:

1. A method for analyzing biological particles in a biological sample analyzer, comprising:
(a) establishing a flow of a fluid medium within a sample inlet tube, the fluid medium carrying a plurality of discrete biological particles, each of the biological particles having at least one face with a substantially planar region;
(b) applying a substantially elliptical acoustic field having a major axis along a direction of a maximum acoustic field strength in a cross-sectional plane of the sample inlet tube, to focus and preferentially orient the biological particles, such that a respective face of each of the biological particles is oriented normal to the major axis; and
(c) analyzing signals received from the preferentially oriented biological particles to identify desired characteristics of the biological particles.

2. The method of claim 1, wherein step (b) comprises:
applying the substantially elliptical acoustic field using a linear transducer coupled to a portion of the sample inlet tube.

3. The method of claim 1, wherein step (b) comprises:
applying the substantially elliptical acoustic field consistently along a substantial length of the sample inlet tube.

4. The method of claim 1, further comprising:
after step (b), introducing a sheath fluid that surrounds the fluid medium carrying the biological particles.

5. The method of claim 4, further comprising:
controlling a pressure applied to the sheath fluid to hydrodynamically focus and maintain the preferential orientation of the acoustically focused biological particles approximately along a central axis of a flow cell.

6. The method of claim 1, wherein the biological particles comprise sperm cells.

7. The method of claim 6, wherein step (c) includes:
identifying DNA content of chromosomes as the desired characteristics of the sperm cells.

8. The method of claim 7, further comprising:
(d) sorting the sperm cells according to the identified DNA content of chromosomes.

9. The method of claim 1, wherein the biological particles comprise red blood cells.

10. The method of claim 1, wherein the biological particles comprise substantially disk-shaped particles, wherein an area of the face of the biological particle is relatively larger than an area of a side surface of the biological particle.

11. A method for orienting biological particles in a biological sample analyzer, comprising:
(a) establishing a flow of a fluid medium within a sample inlet tube, the fluid medium carrying a plurality of discrete biological particles, each of the biological particles having at least one face with a substantially planar region; and
(b) applying a substantially elliptical acoustic field having a major axis along a direction of a maximum acoustic field strength in a cross-sectional plane of the sample inlet tube, to focus and preferentially orient the biological particles, such that a respective face of each of the biological particles is oriented normal to the major axis.

12. The method of claim 11, further comprising:
sorting the biological particles according to desired characteristics identified by analyzing signals received from the preferentially oriented biological particles.

13. The method of claim 11, wherein the biological particles comprise sperm cells.

14. The method of claim 11, wherein the biological particles comprise red blood cells.

15. The method of claim 11, wherein the biological particles comprise substantially disk-shaped particles, wherein an area of the face of the biological particle is relatively larger than an area of a side surface of the biological particle.

16. The method of claim 11, wherein step (b) comprises:
applying the substantially elliptical acoustic field using a linear transducer coupled to a portion of the sample inlet tube.

17. The method of claim 11, wherein step (b) comprises:
applying the substantially elliptical acoustic field consistently along a substantial length of the sample inlet tube.

18. The method of claim 11, further comprising:
after step (b), introducing a sheath fluid that surrounds the fluid medium carrying the biological particles.

19. A method for separating lipid particles from a lipemic serum free of blood cells, comprising:
(a) applying an acoustic field along a length of a sample inlet tube to acoustically drive the lipid particles towards a peripheral region of the sample inlet tube, leaving a lipid-free clear serum in a central region of the sample inlet tube; and
(b) dividing an output end of the sample inlet tube into first and second output channels, wherein the first output channel collects a portion of the serum including the acoustically-driven lipid particles from the peripheral region of the sample inlet tube, and the second output channel collects the lipid-free clear serum from the central region of the sample inlet tube.

20. The method of claim 19, further comprising:
(c) directing the lipid-free clear serum towards an inlet of a chemical analyzer.

21. The method of claim 19, further comprising:
(d) using results of the measurements done by the chemical analyzer for clinical diagnostics.
